(19) United States
(12) Patent Application Publication
(10) Pub. No.: US 2011/0082357 A1
(43) Pub. Date: Apr. 7, 2011

(54) METHOD AND APPARATUS FOR CO2 EVALUATION

(75) Inventor: Ofer Hornick, Zur Igal (IL)

(73) Assignee: NEETOUR MEDICAL LTD., M.P. Hefer (IL)

(21) Appl. No.: 12/993,588

(22) PCT Filed: May 27, 2009
(86) PCT No.: PCT/IL09/00530

§ 371 Date: Nov. 19, 2010

Related U.S. Application Data
(60) Provisional application No. 61/056,452, filed on May 28, 2008.

(51) Int. Cl. A61B 5/00

(52) U.S. Cl. 600/364

(57) ABSTRACT

A method for evaluating CO₂ level in the blood of a patient, comprising detecting in the patient's body at least one haemodynamic signal from at least one tissue or part thereof, processing the at least one haemodynamic signal to derive a value related to the CO₂ level of the patient and determining an evaluation of CO₂ level of the patient based on a relation of the derived value to the CO₂ level of the patient, and an apparatus to carry out the same.

### FIELD OF THE INVENTION

The invention relates to evaluation of CO₂ level in the blood of a patient. Some embodiments of the invention relate to deriving an evaluation of CO₂ level based on non-invasive detection of one or more signals related to haemodynamic parameters.

### BACKGROUND OF THE INVENTION

The level of CO₂ (Carbon Dioxide) in the blood of humans and other beings has several significant biologic functions such as in respiratory rate and depth control, muscle contraction or dilatation of arterioles where, typically, higher resistance is due to vessels constriction and lower resistance is due to vessels dilation.

Clearly, the ability to measure and monitor CO₂ levels are of significant clinical value. Indeed, different methods and devices have been developed for measuring this parameter. Known devices include laboratory tests measuring CO₂ levels in a blood sample, devices testing CO₂ levels directly from an arterial line catheter, capnographs or capnometers that measure CO₂ levels in the exhaled air (generally being in good correlation with blood CO₂ levels) or transcutaneous CO₂ monitors which use heated electrodes attached to the skin, measuring the local carbon dioxide gas tension of the tissue. While these devices may provide valuable information, they are, in general, costly and require disposable elements and some of these devices, such as intra-arterial sensors, are invasive.

While CO₂ monitoring is a major parameter for assessment of breathing, yet under certain clinical circumstances, such as emergency conditions, CO₂ monitoring may be cumbersome. For example, a capnograph cannula attached to the patient's nose may dislodge and fail to provide reliable values.

Methods and apparatus for measurement of CO₂ in patients are disclosed in prior publications, some of which are cited below as examples.

U.S. Pat. No. 6,741,876 relates to measurement of blood constituents, including CO₂, by spectroscopy; US application 2007/0129645 relates to invasively measuring respiration waveform and deducing CO₂ level from the respiratory waveform parameters; U.S. Pat. No. 6,819,950 relates to non-invasive measurement of blood absorption at two locations and deducing CO₂ levels from a pH parameter; U.S. Pat. No. 7,405,055 relates to determination of a blood constituent, including CO₂, using a single device by a particular formula; US application 2007/0027375 relates to non-invasive measurement of blood flow at two locations and deducing CO₂ levels from an average of the measurements; U.S. Pat. No. 5,766,127 relates to simultaneous spectroscopic measurements at about the same location to deduce blood perfusion; U.S. Pat. No. 7,341,560 relates to monitoring blood parameters by a plurality of light sources and detectors positioned on a single body part; U.S. Pat. No. 6,942,622 relates to monitoring the effects of blood/haemodynamic parameters including CO₂ on autonomic tone; U.S. Pat. No. 6,501,975 relates to correlating two blood signals from a single location for deriving blood gas concentration; U.S. Pat. No. 6,826,419 relates to correlating two blood signals from a single location for deriving blood gas concentration; US application 2004/0204638 relates to correlating two blood signals from a single location for deriving blood constituent concentration; U.S. Pat. No. 7,351,203 relates to covariate monitoring at a single location, including monitoring CO₂; US application 2005/0076909 relates to covariate monitoring including CO₂ but no derivation of CO₂; US application 2004/0236240 relates to monitoring respiratory conditions based on blood parameters including CO₂ but no derivation of CO₂; U.S. Pat. No. 7,225,013 relates to using CO₂ signal for predicting change in a patient; U.S. Pat. No. 7,195,013 relates to modulating autonomous function using CO₂ signal; and U.S. Pat. No. 6,896,660 relates to covariate monitoring, including CO₂ as single parameter for estimation of tissue perfusion.

### SUMMARY OF THE INVENTION

Generally, the invention relates to deriving an evaluation of CO₂ level in the blood of a patient by processing of one or more detected signals related to one or more haemodynamic parameters of the patient. Preferably the signals are detected non-invasively.

For brevity and clarity, without limiting and unless otherwise specified, a signal or part thereof related to a haemodynamic parameter, or a signal or part thereof of the haemodynamic parameter, are denoted herein interchangeably as ‘haemodynamic signal’ or ‘haemodynamic waveform’.

Accordingly, a general aspect of the invention relates to a method and apparatus for evaluating CO₂ level of a patient by detecting at the patient's body at least one haemodynamic signal from an at least one tissue (such as an organ or part thereof), processing (employing) the at least one haemodynamic signal to derive a value related to the CO₂ level of the patient, and based on a relation of the derived value to CO₂ determining an evaluation of CO₂ level of the patient, wherein in some embodiments the derived value constitutes the evaluation of CO₂ level.

An aspect of the invention relates to a method and apparatus for detecting at a site of the patient's body a haemodynamic signal from a tissue, processing the waveform and deriving a value functionally related to the CO₂ level of the patient. In some embodiments of the invention, the CO₂ level of the patient is linearly determined from the derived value.

Another related aspect of the invention relates to a method and apparatus for simultaneously detecting haemodynamic signals from a plurality or tissues, processing the signals and deriving a value functionally related to the CO₂ level of the patient based on interrelation between the signals.

In some embodiments of the invention, one site of the patient is used for detection in a plurality of underlying tissues. Optionally and alternatively, a plurality of sites is used for detection in underlying tissues.

In some embodiments of the invention, the interrelation between the signals is due to the physiological differences in the response of vascular beds in different body organs or tissues. While variations of CO₂ levels in most of the blood vessels affect changes of haemodynamic parameters in a certain direction, variations of sympathetic nervous system activity affect changes in opposite directions in different organs (such as muscle versus skin) and changes of a different magnitude in other organs (such as brain).

In some embodiments of the invention, evaluation of CO₂ level based on the simultaneous correlation between haemodynamic parameters may provide a better performance in terms such as precision and/or repeatability and/or consistency between patients and/or reliance on calibration relative to an evaluation based on a single parameter, while the interrelation between the simultaneously detected signals can be used to assess the activity of the autonomic nervous system.

In some embodiments of the invention, the CO₂ level is evaluated periodically, optionally providing continuous monitoring of the CO₂ level of a patient.

In some embodiments, the detectors are connected to or integrated with other components providing a system (apparatus) for evaluation and/or monitoring of CO₂ levels of a patient and optionally for performing other activities such as derivation and calculations of other parameters of the patient, archiving, trending, correlation and linkage with other systems.

In some embodiments of the invention, the system comprises or is linked with a processor and comprises or is linked with a medium comprising or storing a program that implements an algorithm for processing the acquired signals and performing the computations to obtain a value of the CO₂ level of the patient. Typically and optionally, the system comprises or is linked with a medium comprising or storing a program that controls the signal detection and/or operation interface or any designed activity.

Any adequate new or customized or other equipment suitable for detecting and acquiring haemodynamic signals may be used. Some detectors for acquiring haemodynamic signals are known in the art, including standard (off-the-shelf) devices and including non-invasive devices. For example, non-invasive detectors such as transcranial Doppler ultrasound probes (TCD) for detecting flow in brain vessels or IR/visible light Photoplethysmography (PPG) probes or oximeters, wherein the standard equipments is, optionally, modified or adjusted.

In some embodiments, the detected signals are optionally used to obtain other values in addition to and as complementary values to CO₂ evaluation, whether by known methods and/or devices of the art or modifications thereof or by new methods and/or devices. For example, other haemodynamic measurements, heart rate, blood oxygen saturation (SpO₂), respiratory depth, respiratory rate and variability, blood pressure and variations thereof, or heart rate and variability thereof. The other values may also be used for assessment of the patient condition and/or adjusting or correction of the CO₂ evaluation.

In the specification and claims the following terms and derivatives and inflections thereof imply the respective non-limiting characterizations below.

Patient—humans and other non-human mammals.

CO₂ level in the blood (of a patient)—CO₂ partial pressure in the blood or an approximation thereof sufficiently close to indicate a clinical state or a physiological state. For example, as a correlation with EtCO₂ of a capnometer or with direct measurement of blood samples such as by intra-arterial CO₂ analyzer.

Haemodynamic (signal, parameter)—relating to blood flow in a blood vessel or vessels of an organ or tissue or part thereof. For example, resistance to blood flow or mathematical indices correlated with resistance (e.g. pulsatility index (PI), resistivity index (RI), S/D systolic to diastolic ratio (S/D), blood flow velocities), or other mathematical indices correlated with flow or resistance or derivation and/or combination thereof.

Tissue—a tissue or part thereof of the patient's body or some organ or part thereof.

Site (of a patient)—location in or on the body of the patient, such as a patch or region of skin or a portion of muscles.

Waveform/curve—representation of variations of a signal or data, or part thereof (not precluding intervals with constant signal or data).

Signal—values representing some physical or physiological phenomenon, typically in a digital form as a series of numerical values.

Acquisition/detection (of signal)—obtaining a signal via a detector (sensor) in a form suitable for processing, typically as a series of numerical readings accessible to a processor. For example, an analog signal from a sensor, subsequently converted to digital form (ADC).

Detector/sensor—a device or other equipment used to acquire biological signal or signals. Unless otherwise specified or evident from the context, the terms ‘detector’ and ‘sensor’ may be used interchangeably and irrespective if a basic component or a sub-unit of a system is referred to.

According to the context and without limiting, an acquired signal or part thereof (e.g. for a certain time span) is denoted as ‘signal’.

According to the context and unless otherwise specified, a cardiac cycle or a signal of a cardiac cycle or a representation thereof is denoted as ‘cycle’.

Unless particularly indicated, the terms ‘resistance’ and ‘compliance’ are used herein interchangeably denoting blood flow parameters.

According to an aspect of some embodiments of the present invention there is provided a method for evaluating CO₂ level of a patient, comprising:
(a) detecting on the patient's body at least one haemodynamic signal from at least one tissue or part thereof;
(b) processing the at least one haemodynamic signal to derive a value related to the CO₂ level of the patient; and
(c) determining an evaluation of CO₂ level of the patient based on a relation of the derived value to CO₂ level of the patient.

In some embodiments, detecting is performed non-invasively.

In some embodiments, the at least one haemodynamic signal from at least one tissue or part thereof constitute one signal from one tissue or part thereof.

In some embodiments, the at least one haemodynamic signal from at least one tissue or part thereof constitute a plurality of signals from a plurality of similar tissues or parts thereof.

In some embodiments, the plurality of signals are detected substantially simultaneously.

In some embodiments, the similar tissues are disjoint skin regions.

In some embodiments, the at least one haemodynamic signal from at least one tissue or part thereof constitutes a plurality of signals from one tissue or part thereof.

In some embodiments, the plurality of signals are detected substantially simultaneously.

In some embodiments, the one tissue or part thereof is a skin region.

In some embodiments, the at least one haemodynamic signal from at least one tissue or part thereof constitutes a plurality of signals from a plurality of different tissues or parts thereof.

In some embodiments, the plurality of signals are detected simultaneously.

In some embodiments, the plurality of different tissues comprises at least one tissue selected from skin, muscle or brain.

In some embodiments, the plurality of different tissues comprises at least two tissues selected from skin, muscle or brain.

In some embodiments, processing comprises identifying a region on the at least one signal, or a derivation thereof, by which a value functionally related to CO₂ level of the patient is derived.

In some embodiments, identifying a region comprises analyzing a temporal derivative, or a combination thereof, of the at least one signal or a derivation thereof.

In some embodiments, a value functionally related to CO₂ level of the patient is derived by integrating the temporal derivate, or a combination thereof, about the region.

In some embodiments, a value functionally related to CO₂ level of the patient is linearly related to CO₂ level of the patient.

In some embodiments, wherein processing comprises:
(a) defining a model of a haemodynamic parameter based on a plurality of signals from a plurality of different tissue of part thereof; and
(b) substituting in the model at least one separately acquired haemodynamic parameter thereby deriving a value related to the CO₂ level of the patient.
In some embodiments, a value related to the CO₂ level of the patient constitutes the evaluation of CO₂ level of the patient.

According to an aspect of some embodiments of the present invention there is provided an apparatus for evaluating CO₂ level of a patient, comprising:
(a) at least one detector at the patient's body for detecting at least one haemodynamic signal from an at least one tissue or part thereof; and
(b) a processor and a program for deriving an evaluation of the CO₂ level of the patient based on the at least one haemodynamic signal.

In some embodiments, the apparatus further comprises apparatus for providing at least the evaluation of the CO₂ level of the patient.

In some embodiments, the evaluation of the CO₂ level is provided continuously in real-time.

In some embodiments, the at least one detector is non-invasive to the patient.

In some embodiments, the apparatus is sufficiently small and lightweight for wearing by the patient. In some embodiments the apparatus is sufficiently mobile to be worn by an ambulatory patient.

In some embodiments, the apparatus is configured to implement the methods described above.

### BRIEF DESCRIPTION OF THE DRAWINGS

Some non-limiting exemplary embodiments of the invention are illustrated in the following drawings.

Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

FIG. 1 illustrates a chart of a waveform of variations of skin blood vessels pulsatility.

FIG. 2 illustrates a flowchart schematically outlining actions for deriving CO₂ levels from a haemodynamic waveform, according to exemplary embodiments of the invention;

FIG. 3 illustrates a flowchart outlining actions for deriving CO₂ levels from a haemodynamic waveform, according to exemplary embodiments of the invention;

FIG. 4 illustrates aligned and superimposed normalized heart cycles derived from the waveform such as of FIG. 1, according to exemplary embodiments of the invention;

FIG. 5 illustrates the aligned and superimposed first temporal derivatives of normalized heart cycles of a waveform such as of FIG. 1, according to exemplary embodiments of the invention;

FIG. 6 illustrates a representative first temporal derivate of normalized heart cycles of a waveform such as of FIG. 1, according to exemplary embodiments of the invention;

FIG. 7 illustrates a chart of correlated waveforms of evaluated CO₂ levels, EtCO₂ from a capnograph and respiration rate from a capnograph, according to exemplary embodiments of the invention;

FIG. 8 illustrates a chart of statistical correlation between evaluated CO₂ levels and EtCO₂ from a capnograph, according to exemplary embodiments of the invention;

FIG. 9 illustrates a chart of a Bland-Altman agreement analysis between evaluated CO₂ levels and EtCO₂ from a capnograph, according to exemplary embodiments of the invention;

FIG. 10 schematically illustrates a diagram describing how CO₂ levels correlate with skin resistance and muscle resistance, according to exemplary embodiments of the invention;

FIG. 11 illustrates a flowchart schematically outlining actions for deriving CO₂ levels from a plurality of haemodynamic signals, according to exemplary embodiments of the invention;

FIG. 12 schematically illustrates a diagram of CO₂ evaluation system, according to exemplary embodiments of the invention; and

FIG. 13 illustrates a flowchart outlining actions for user operation involved in evaluating CO₂ level of a patient, according to exemplary embodiments of the invention.

### DESCRIPTION OF EMBODIMENTS OF THE INVENTION

The following description relates to one or more non-limiting examples of embodiments of the invention. The invention is not limited by the described embodiments or drawings, and may be practiced in various manners or configurations or variations. The terminology used herein should not be understood as limiting unless otherwise specified.

The non-limiting section headings used herein are intended for convenience only and should not be construed as limiting the scope of the invention.

### Single Signal

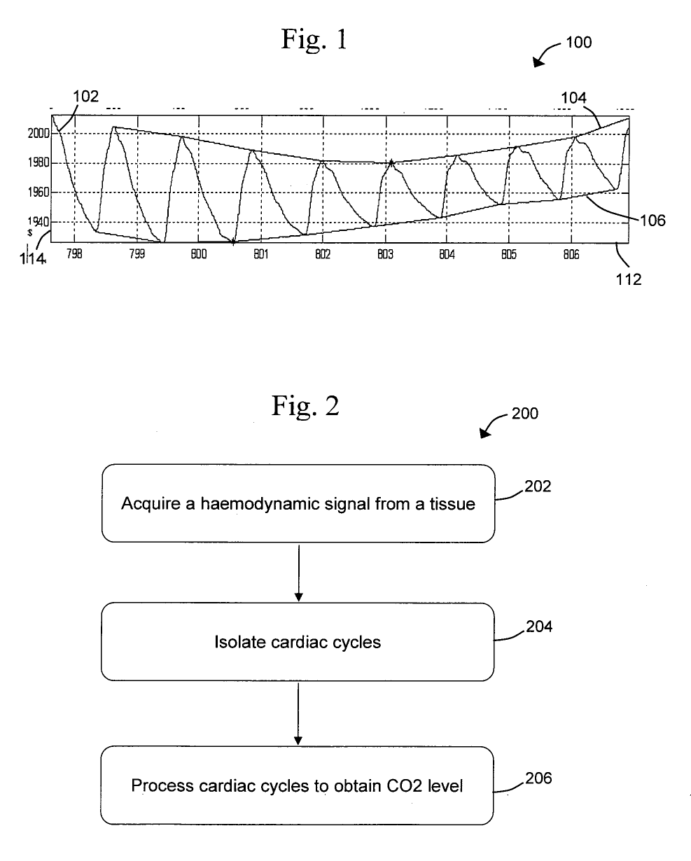

FIG. 1 illustrates a chart 100 of a waveform 102 of variations of blood flow phenomena acquired at a particular tissue (for example, skin) by a detector (for example, PPG), generally representing other haemodynamic signals of a patient.

The horizontal axis 112 denotes a time scale (in seconds) and the vertical axis 114 denotes a scale of the pulsatile phenomena, such as voltage or current at the detector.

Waveform 102 follows (possibly with some delay) the heart cycle (beats) and is modulated by the respiration as exemplified by an envelope of the extremum points of waveform 102 with upper part 104 (maximums) and lower part 106 (minimums).

FIG. 2 illustrates a flowchart 200 schematically outlining actions for deriving CO₂ levels from haemodynamic waveforms, such as 102, according to exemplary embodiments of the invention.

A haemodynamic signal such as waveform 102 is acquired (202), for example via a PPG probe on the skin. In some embodiments, a limited time span of the signal is stored in a memory for subsequent processing.

The acquired signal is analyzed to isolate separate cardiac cycles (204). A plurality of cardiac cycles may be combined (e.g. by averaging), possibly after normalization to a common scale, to represent a typical cycle or cycles of the signal.

The cardiac cycles, or combined cycles as a representative cycle, are processed (206) to obtain CO₂ levels. In some embodiments of the invention, characteristics of the cardiac cycle shape are determined and processed to derive a value functionally related to the CO₂ level, and the CO₂ level is obtained by applying the appropriate formula. Typically the function is a linear formula where, optionally, the coefficients are preset or predefined or obtained by a calibration procedure.

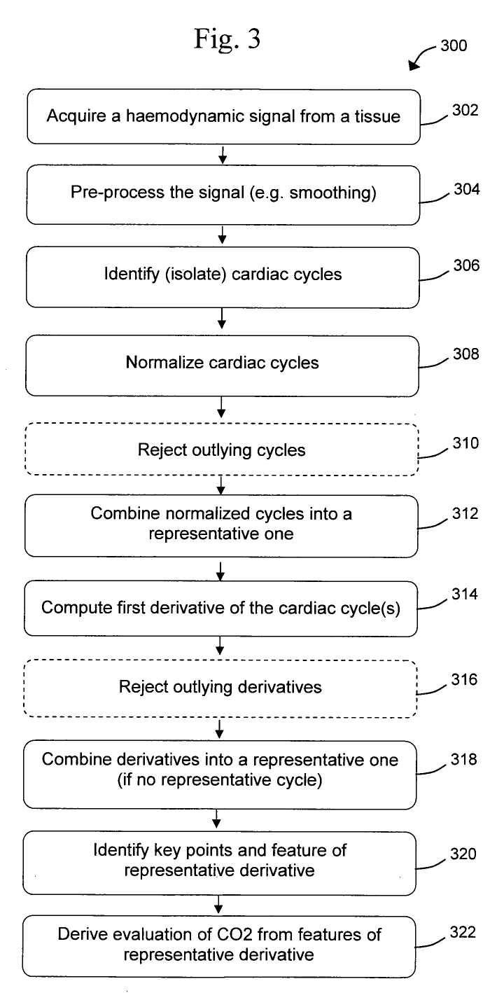

Until otherwise stated, the following discussions below refer also to FIG. 3 that illustrates a flowchart 300 outlining actions for deriving CO₂ levels from a haemodynamic waveform, according to exemplary embodiments of the invention.

### Signal Acquisition

A signal is acquired (302) for a time span comprising a series of several consecutive cardiac cycles, typically but not necessarily covering a respiratory cycle (typically of about 6 seconds). In some embodiments, the cardiac cycles are distinguished, for example, by rough detection of peaks and/or valleys, or by estimated or measured heart rate or by other methods such as estimation based on a previous acquisition. In some embodiments, the acquisition time span is, about 6 or more seconds (e.g. 8 or 12 seconds).

In some embodiments of the invention, the signal, or part thereof, is preprocessed (304) such as by smoothing (e.g. by a low pass filter) to remove noise or other high-frequencies (e.g. spikes) relative to what is expected. Optionally, other signal conditioning is used such as known in the art, for example, exponential filter.

### Cycle Separation

The signal is analyzed to identify and separate the cycles (306), such as by identifying maximum (peaks) and minimum (valleys) regions or points and/or minimal rise and/or descent rates and/or by using signal analysis algorithms of the art.

The separated cycles, or sub-set of the cycles, are normalized (308) to a common scale such as by scaling them so that the peaks share a common value (e.g. 1) and the valleys share a common value (e.g. 0) and, optionally, all the cycles start at a common virtual time such as t=0. Optionally the cycles' widths are adjusted to share a common or approximate common width such as to compensate for varying heart rate.

For example, referring to waveform 102 of FIG. 1, the envelope of extremum points (104 and 106) may be evaluated or approximated by a function or series of functions such as spline or splines and/or a polynomial formula or formulas (e.g. of the 3^{rd} degree or higher), optionally taking into account a full breathing cycle (or cycles) and effects thereof on the cardiac pulse signal. In some cases a sufficient approximation is a series of lines connecting the extremum points.

For each cycle the respective lower envelope 106 is subtracted and the result is divided by the resultant maximal values, providing cycles in a 0-1 range.

Before or after the normalization, the cycles are analyzed to reject (ignore or discard) outliers (310), such as cycles that do not fit the expected and/or predefined or determined (e.g. learned) constraints and/or the general shape of the majority of the cycles, such as artifacts or distorted shapes due to the patient condition or movements. In some embodiments, the rejection is based on median filter or properties of the cycles such as area or height or width or rate of change, or the rejection may be based on other methods of the art.

Having ignored the rejected cycles, in some embodiments of the invention the cycles are used to obtain a representative cycle or cycles of the time span (312). For example, a typical cycle or resembling cycles are selected or a combination of the cycles is used as a representative cycle (see more below).

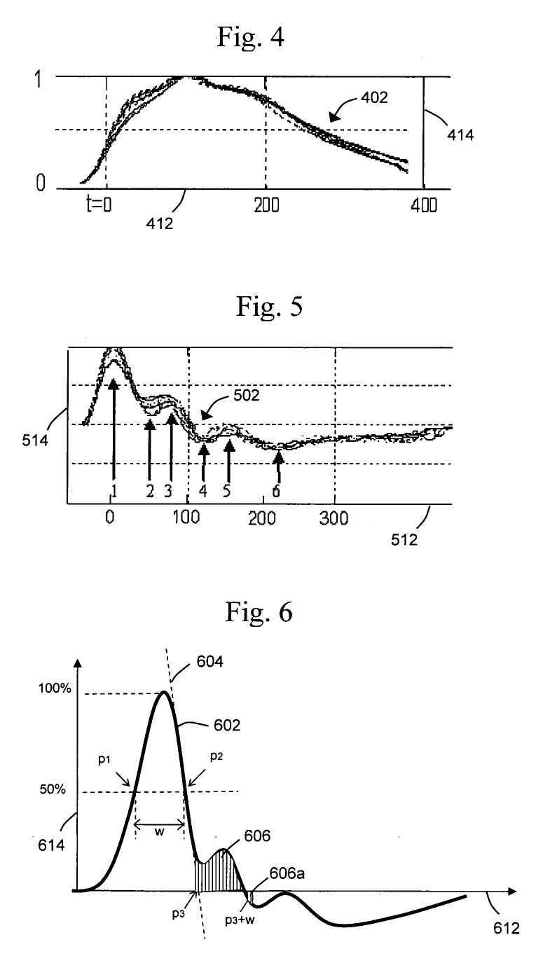

FIG. 4 illustrates aligned normalized heart cycles 402 derived from a waveform such as waveform 102 of FIG. 1. At the vertical scale 414 the cycles' peaks are set at a level of 1, the bases at a level of 0 and the cycles are aligned and superimposed on each other and with respect to time scale 412 such that the maximum points of the first derivate vs. time (temporal derivative) or the peaks of the cycles are set at t=0. Optionally or alternatively, in some embodiments, the cycles' peaks or derivatives maximal points are aligned at a common arbitrary virtual time.

In some embodiments of the invention, the aligned cycles, having a common scale and time (and optionally approximately common width) are added up and divided by the number of cycles to obtain a representative cycle (simple average). Optionally or additionally, a weighted average is performed where cycles that deviate from the majority of the cycles and/or from the simple average such as by area difference are given lower weight relative to cycles that deviate less, optionally functionally related to the difference. Optionally or alternatively, other methods are used to obtain representative cycle or cycles such as by picking cycles that have the largest correlations between the cycles.

In some embodiments of the invention, the assemblage of normalized cycles, or alternatively one or more representative cycles are further processed.

For brevity and clarity, relating to the cycles in the discussions below implies either an assemblage of the normalized cycles or one or more representative cycles thereof, unless otherwise specified or evident from the context.

### Shape Analysis

In some embodiments, the shapes of the cycles are further analyzed by taking the first temporal derivate of the cycles (‘the derivative’) (314).

FIG. 5 illustrates the aligned and superimposed first temporal derivatives 502 of normalized heart cycles of a waveform such as waveform 102 of FIG. 1. With respect to magnitude scale 514 the maximal points (peaks) of the derivates are aligned a at virtual time t=0 of time scale 512.

Typically, several zones are discerned in the derivative shape, as listed in Table 1 below (and with respect to FIG. 5 that shows corresponding numerals):

TABLE 1

| Numeral | | Approximate typical |
|---|---|---|
| label | Zone | time (ms) |
| 1 | First maximum point | 0 |
| | (global maximum) | |
| 2 | First Minimum point | 50 |
| 3 | Second maximum | 80 |
| | (alternatively as an | |
| | inflection point) | |
| 4 | Second minimum | 125 |
| 5 | Third maximum point | 150 |
| 6 | Third minimum point | 220 |

In some embodiments, before further analysis, the derivates are pre-processed including, without limiting, the following steps:
Rejection (ignoring or discarding) of outliers (316), such as derivative signals that do not fit the expected and/or the general shape of the majority of the cycles. In some embodiments, the rejection is based on median filter of properties of the signals such as area or height or width of the derivatives signals 502 that do not conform to a predefined or determined (e.g. learned from pervious or other measurement) set of constraints. Optionally, in some embodiments, the rejection is based on the values and/or separation in time of the points in derivates 502 as listed in Table 1, such as first maximal (global) maximum (1) or third minimum (6). For example, if the separation is more or less by 30% of the expected separation. Optionally or additionally, the rejection may be based on other methods of the art. In case of a single representative cycle this instant step is immaterial.
Smoothing the retained (non-rejected) derivates, such as by a low pass filter to remove noise such as due to derivative properties or to remove residual effects of breathing.

The shapes of derivatives 502, or selected typical derivatives shapes, are combined (e.g. average, weighted average, median selection) to form a representative derivative shape (318) (unless a single representative shape was previously obtained and the derivate of which was taken). In order to reduce sensitivity to variations and possible distortions in the signals, in some embodiments derivates 502 are selected within a significantly longer time span than a typical respiration cycle (e.g. several respirations cycles such as 30 or 60 seconds) or from several acquisitions.

FIG. 6 illustrates a representative first temporal derivate 602 of normalized heart cycles of a waveform such as waveform 102 of FIG. 1 (hereinafter, also ‘ShapeD’). The illustration is with respect to relative magnitude scale 614 and time axis scale 612 (similar to time scale 512 of FIG. 5), wherein the maximal value (‘1’ in FIG. 5) is taken as 100%. FIG. 6 also illustrates auxiliary lines and features (e.g. ‘p1’, ‘w’) to further clarify the discussion below and reference to FIG. 6 is accordingly implied.

Representative first temporal derivate ShapeD is further analyzed to obtain key points and features in ShapeD (320) as follows:
Determine the points in ShapeD where the initial (temporal, time-wise) ascent and descent are at 50% of the peak (100%), namely, p1 and p2, respectively. Optionally or alternatively, instead of using the 50% level, the inflection point level of the rise or fall, or combination thereof is used (such as by averaging or time-wise distance between the inflection points).
Calculate the time-wise distance between points p1 and p2 (hereinafter, ‘wid’ equivalent to ‘w’ in FIG. 6).
Determine the tangent 604 to the initial temporal descent at point p2.
Determine the intersection of tangent 604 with the time axis 612 to obtain intersection point p3.
Compute the integral between ShapeD and time axis 612 between intersection point p3 and p3+wid (timewise), shown as striped region 606 and 606a (collectively 606). Since ShapeD is a representative first derivate of the normalized heart cycles, integral 606 is equivalent to the difference between the normalized cycle between corresponding point p3 and p3+wid (corresponding on the time axis 412 with respect to one or combined curves in FIG. 4).

A possible rationale behind the above procedure is to calculate a normalized value from a cycle, where this value represents the decay of the heart cycle signal, from the “expected maximum point” represented as point p3.

It was unexpectedly found that the value of integral 606 (hereinafter also ‘AreaD’) tracks, at least approximately, the CO₂ level, (and may be regarded also as haemodynamic parameter or index)

### CO₂ Evaluation Derivation

In some embodiments of the invention, CO₂ level (‘CO₂L’), at least with an approximate relation to a capnograph, is derived from AreaD (322) as follows.

The functional expression for obtaining CO₂L is expressed as:

CO₂L=M×AreaD+N (1)

In some embodiments, a sufficiently (such as of clinical significance) approximation is achieved by setting coefficient ‘M’ as M=80. Optionally, other values are used, optionally or additionally, by determining or adjusting coefficient ‘M’ according to previous measurements or other references such as blood samples.

In some embodiments, coefficient ‘N’ can be derived by calibration of CO₂L relative to a reference such as a capnograph or according to blood samples or intra-arterial CO₂ analyzer. Optionally or alternatively, CO₂L is calibrated assuming a normal physiology and/or condition of the patient which can be monitored and assessed according to the signals (such as 402 of FIG. 4 or 502 of FIG. 5). Normal physiology and/or condition, which may also be obtained by using the same detection apparatus or an auxiliary detection apparatus, are, for example, normal breathing (e.g. about 6 seconds per cycle), normal heart rate (e.g. about 60-70 bps) or normal SpO2, or combinations thereof. Assuming CO₂L in normal conditions to be about 38 mmHg, coefficient ‘N’ is obtained from formula (1) by:

N=CO₂L−M×AreaD (2)

In some embodiments of the invention, coefficient ‘N’ is adjusted or determined periodically or responsive to perceived (detected) change of the patient condition, and some previously determined values of CO₂L may be used as in formula (2) above.

In some embodiments of the invention, one or more of the coefficients ‘M’ and ‘N’ may be obtained by comparing and/or correlating the detected signal (such as waveform 102) to a typical or representative corresponding detected signal, or by comparing and/or correlating ShapeD to a typical or representative derivative of CO₂ signal in a normal or typical patient. See also discussion on using templates and limits below.

In some embodiments of the invention, a better accuracy of and/or sensitivity to CO₂ levels are achieved by non-linear formulas or other methods (e.g. fuzzy logic) and the parameters of the formulas (e.g. polynomial or exponent) or settings of the methods are calibrated and adjusted similarly as described for formulas (1)-(2). The non-linear computation is, in some embodiments, beneficial relative to the linear computations in cases of seemingly non-realistic high and/or low CO₂ levels that were derived linearly such as by formulas (1)-(2) above.

### Experimental Results Example

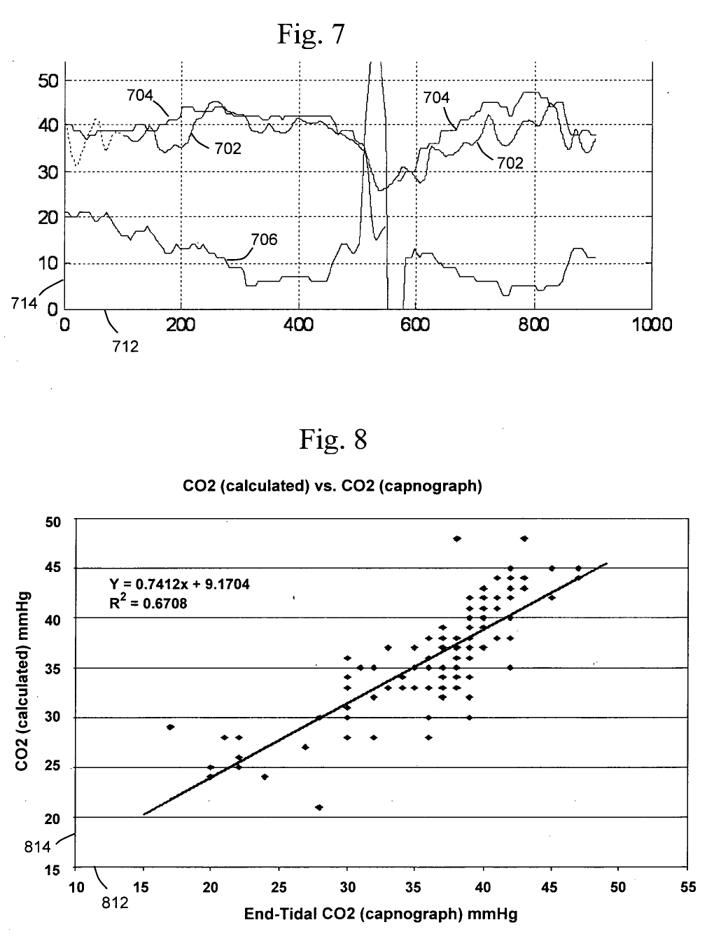

FIG. 7 illustrates a chart, with vertical scale 714 of CO₂ level in mmHg and with horizontal scale 712 in virtual time in seconds, of correlated waveforms of evaluated CO₂ levels 702, EtCO₂ from a capnograph 704 and respiration rate from a capnograph 706, according to exemplary embodiments of the invention.

As can be seen in FIG. 7, evaluated CO₂ level 702 approximately corresponds to EtCO₂ level 704, with maximal deviation of less than about 8 mmHg.

FIG. 8 illustrates a chart, with vertical scale 814 of CO₂ level valuation 814 in mmHg and with horizontal scale 812 of capnograph EtCO₂ in mmHg, of statistical agreement between evaluated CO₂ levels and EtCO₂ from a capnograph, according to exemplary embodiments of the invention.

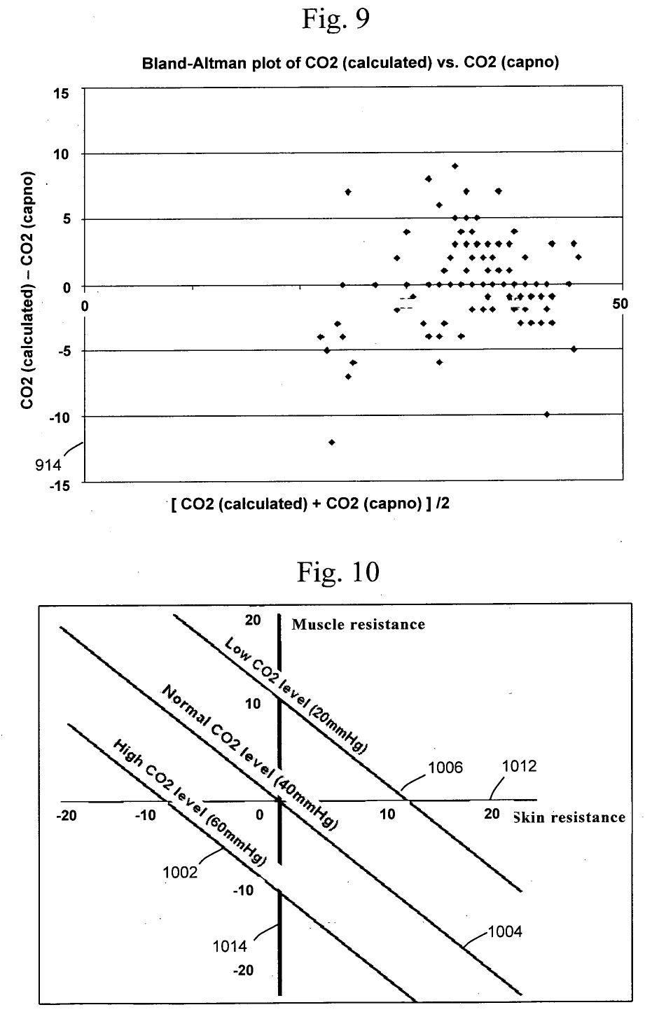

FIG. 9 illustrates a chart of a Bland-Altman correlation between evaluated CO₂ levels and EtCO₂ from a capnograph, according to exemplary embodiments of the invention.

The average difference between linearly derivedCO₂ as described above and CO₂ from a capnograph is 0.29 which is clinically sufficiently small positive bias, and the Standard deviation of the differences is 3.09. In interpreting Bland-Altman plots, it is expected that the majority of data points would fall between the lines denoting 2StD above and below the zero line as FIG. 9 indeed illustrates.

Unless otherwise stated, no further reference to FIG. 3 is implied.

### Enhancements

In some embodiments of the invention, the derived CO₂L is correlated with other measurements, such as PPG at muscle sensor, respiration rate, respiration depth, heart rate variability or heart rate to validate and/or adjust the CO₂L derivation.

In some embodiments of the invention, the method described above for obtaining CO₂L level based on AreaD, or a similar method to that effect, can be simultaneously applied to another similar tissue or tissues (e.g. other skin regions/patches) to obtain additional simultaneous CO₂L values. Subsequently the plurality of AreaD values and/or CO₂L values may be manipulated (e.g. combined, averaged) to obtain CO₂ evaluation of the patient with higher fidelity relative to a single tissue. See also discussion below with respect to a plurality of tissue. In some embodiments, different sensors are applied simultaneously to the same tissue (e.g. particular skin patch or region such as a finger tip) and the signals and/or derived values are manipulated or combined such as by correlation or averaging or by other methods such as weighted average to obtain CO₂ evaluation with higher fidelity relative to a single sensor.

It should be noted that using AreaD is an example of obtaining a quantity related to CO₂ level based on analysis of the signal or derivative or other derivation thereof, and other methods may be used to obtain quantities related to CO₂ levels, possibly correlated with physiological activities.

### Plurality of Signals

In some embodiments of the invention, in order to improve the accuracy of the evaluation of CO₂, notably under some particular physiological or clinical conditions, a plurality of tissues are detected simultaneously for a plurality of signals related to haemodynamic parameters and the interrelations between the signals (or derivations thereof) is used to derive an evaluation of CO₂ level in a patient.

The interrelations between the signals is based on the physiological differences in reactions of vascular beds in different body organs to CO₂ levels vs. reactions to other effectors, such as autonomic nervous system activity. While changes in CO₂ levels cause changes in same direction in most body blood vessels, changes of sympathetic nervous system activity cause changes in opposite directions and different magnitudes in different organs (such as muscle versus skin) and changes of a different magnitude in other organs (such as the brain).

### Possible Mechanisms

A possible explanation to the different haemodynamic behavior of different tissues is that the diameters of arteries change in response to some of the following stimuli:

Neural—Activity of the autonomic nervous system (Sympathetic and Parasympathetic divisions) that respond to a number of external and internal changes, epinephrine for example.

Chemical—response to changes in blood levels of several chemicals, including CO₂ in particular and others such as lactic acid, angiotensin, oxygen and NO.

Some stimuli are systemic (autonomic activation, blood CO₂ levels, blood pressure changes or endocrine control) while others may be local such as local release of endothelial factors due to various events possibly including exercise, with possible further downstream effects, or local neurogenic reflexes and para-endocrine control.

Generally, the hemodynamic changes are not specific to the type of stimulus, and they sum-up to constriction/dilatation of the blood vessel thereby raising/lowering resistance to blood flow, changing blood pressure, and/or decreasing/increasing blood flow. A complex interaction may occur between the stimuli. For example, while CO₂ levels rise, the blood vessel dilates yet rising CO₂ levels beyond a certain threshold may also act on the vasomotor center in the brainstem to activate the sympathetic system, which in turn will counteract the vasodilation and constrict the vessel (such as in the skin) or may further dilate it (such as in a muscle). Sympathetic activity also acts on the heart to increase heart rate, stroke volume and cardiac output, and the increased blood flow may affect blood flow waveforms in arteries.

Based on recognition of the different response to stimuli (e.g., autonomic system and CO₂ levels) as described above, in some embodiments of the invention, the simultaneous changes in different vessels is processed and, based on mathematical equations, the level of blood CO₂ is evaluated.

For simplicity and clarity, the descriptions below provide examples in linear terms which are valid for certain inter-relationships or conditions. Yet, it should be understood that for complex interactions such as described above, the overall behavior should be described in more elaborate terms such as non-linear formulas.

Some embodiments of the invention are based on the understanding that during most cases of clinical patient monitoring, the patient has to remain quiescent. Consequently, it is expected that the major impact on blood flow are due to CO₂ and autonomic function while other factors are estimated to be either of negligible impact or affect the vascular system in the same direction and magnitude, such that the signals and derived evaluation of CO₂ are not detrimentally affected. For example, while a CO₂ rise brings about vasodilatation in most of the human body arteries (except for pulmonary arteries at certain situations), activation due to stimuli of the sympathetic system will produce vasodilation in muscle arteries, and at the same time constriction of blood vessels to the skin, kidneys and other organs while having a minimal influence on brain blood vessels. The following Table 2 summarizes a simplified representation of changes described above:

TABLE 2

| | Para-Sympathetic | Sympathetic | |
|---|---|---|---|
| | activation | activation | CO₂ Increase |
| Skeletal muscle | Constrict | Dilate | Dilate |
| Skin | Dilate | Constrict | Dilate |
| Brain | Minor effect | Minor effect | Dilate |

It should be noted that Table 2 merely shows a simplified representation of the physiological effects. For example, when CO₂ levels go above or below a known threshold level, reflex sympathetic activity may occur. However, this sympathetic activity might have effects in the same direction noted in the table while the change in CO₂ levels may maintain effects attributed to CO₂. Therefore, for blood vessels in some organs the sympathetic reflex may diminish the effects of CO₂, while in others the same reflex may enhance the CO₂ effect.

It should also be noted that some of the changes outlined above are immediate and are subsequently compensated by tissue auto-regulation mechanisms. The compensation mechanism implies that initial flow changes are compensated quickly and flow may return to normal within a very short time after a change in sympathetic activation. The compensatory change, however, involves a change in the overall resistance and compliance of the local vasculature, a change that is manifested in the haemodynamic indices, as measured and calculated by the methods described herein. The quick variations noted above are with respect to duration of one or few heart beats or a respiration cycle.

### Exemplary Arbitrary Units

For simplicity and clarity, the impacts on the autonomic system will hereinafter be referred to as the combined sum of activities thereof (sympathetic and parasympathetic). A maximal arterial dilatation (loss of smooth muscle tone) will receive the value of −10, while maximal constriction will receive the value of +10. Each division of the autonomic system will receive a number from 0 to 10 to represent the activity of the respective division. The Table 3 below represents the arterial smooth muscle tone, on a scale from −10 to +10, as a result of different combinations of sympathetic and parasympathetic activations in a theoretical physiology where CO₂ effect is non-existent and wherein Arterial Tone is equal to Autonomic Tone.

TABLE 3

| Sympathetic | Parasympathetic | Arterial Autonomic′ |
|---|---|---|
| Tone | tone | Tone |
| 10 | 0 | 10 |
| 10 | 5 | 5 |
| 10 | 10 | 0 |
| 5 | 0 | 5 |
| 5 | 5 | 0 |
| 5 | 10 | −5 |
| 0 | 0 | 0 |
| 0 | 5 | −5 |
| 0 | 10 | −10 |

Having a scale for autonomic activity on blood vessel diameter/resistance arbitrarily defined between +10 (complete dilatation in skeletal muscle arteries) and −10 (complete constriction in skeletal muscle arteries), similarly the effect of CO₂ on blood vessels is herein defined using a similar scale, from +10 (complete dilatation effect when CO₂ levels are maximal) to −10 (complete constriction effect when CO₂ levels are minimal).

### CO₂ Derivation Overview

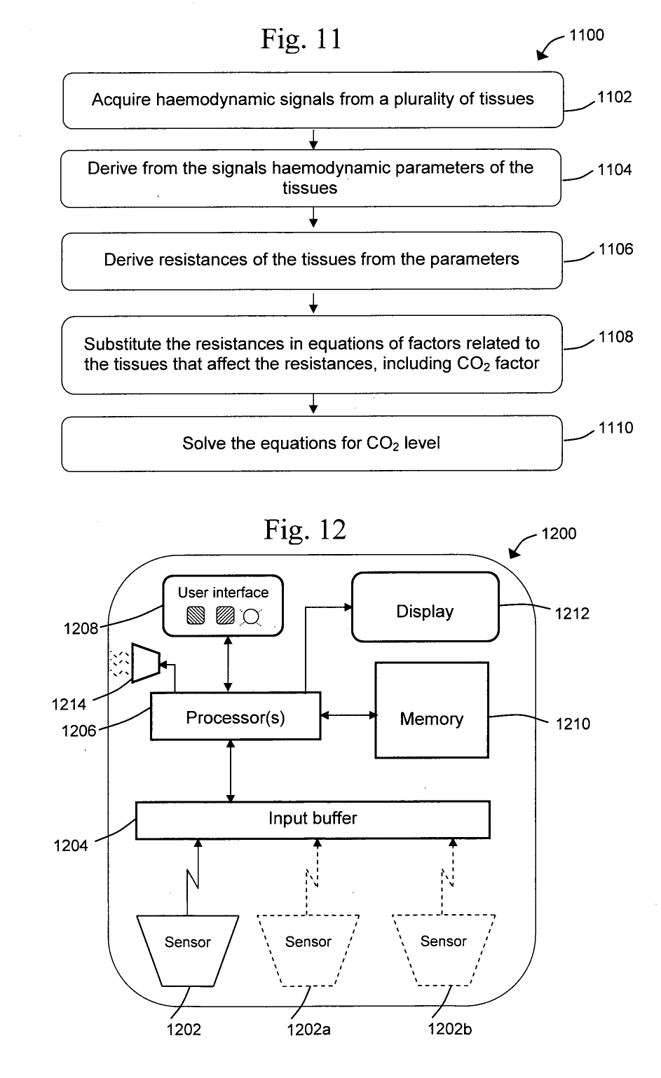

FIG. 11 illustrates a flowchart 1100 schematically outlining actions for deriving CO₂ levels from a plurality of haemodynamic signals, according to exemplary embodiments of the invention.

Haemodynamic signals from a plurality of tissues, such as skin, muscle or brain, are acquired (1102).

Haemodynamic parameters of the tissues, such as PI, RI, V or S/D are derived from the signals (1104). A haemodynamic parameter can also be derived as described, for example, for AreaD above, or other haemodynamic parameters may likewise be derived. For different tissues the same or different haemodynamic parameters can be used, as well as combinations of different parameters.

Resistances of the tissues are derived from the haemodynamic parameters according to methods such as known in the art (1106).

The derived resistances of the tissues are substituted in the equations of factors related to the tissues that affect the resistances (interaction model), including CO₂ factor and autonomous system factor (1108).

### Exemplary Model

An exemplary, simplified for clarity, non limiting mathematical model that portrays how both factors, namely, autonomic and CO₂ level, interact on the blood vessel and affect the total resistance of the vessels to blood flow is formulated below (formulas (3)-)(5)). It should be noted that other, possibly more elaborate, models, may be used.

RES(muscle)=F(A(mcl)×CO₂+B(mcl)×Aut+C(mcl)×Oth+D(mcl)) (3)

RES(skin)=F(A(skin)×CO₂+B(skin)×Aut+C(skin)×Oth+D(skin)) (4)

RES(brain)=F(A(brn)×CO₂+B(brn)×AuT+C(brn)×Oth+D(brn)) (5)

Where:

F is a function of the arguments;

RES (organ) is the total combined resistance/compliance of blood vessels in the respective organ;

A (organ) is a coefficient describing the relationship between CO₂ level (denoted in the model as ‘CO₂’) and the effect thereof on the respective organ;

B (organ) is a coefficient describing the relationship between Autonomic activity level (‘Aut’) and the effect thereof on the respective organ;

C (organ) is a coefficient describing the relationship between levels of other additional factors or stimuli (‘Oth’) in addition to CO₂ and Autonomic activity, and the effect thereof on the respective organ. C (organ) may be replaced by particular coefficients related to specific factors.

D (organ) is a constant factor related to intrinsic features of the blood vessels in the respective organ without external effect.

For brevity and clarity, ‘muscle’ is abbreviated to ‘mcl’ and ‘brain’ to ‘brn’.

At least for an approximation, the function ‘F’ is considered to be a unity, namely, formulas (3)-(5) are linear formulas.

The equations and coefficients may be defined differently at different ranges of physiological parameters. For example, A (organ) may have a value A₁ in a range of 0-30 mmHg CO₂, a value A₂ in a range of 30-45 mmHg and a value A₃ above 45 mmHg, yet within a specified range, a set of constant coefficients applies.

A likely underlying assumption in some embodiments of the invention is that besides autonomic function and CO₂ levels, the effects of other factors are maintained constant, at least approximately, under monitoring conditions. As patients usually remain at rest or are required to do so, and as many of the other factors change due to physical activity or to local circulatory conditions, the assumption is likely to be valid under most clinical conditions. It is also assumed that other effects (in addition to CO₂ and autonomic activation) either change in the same magnitude and direction, or are of negligible magnitude, so the effects are cancelled in formulas (3)-(5). The existence of other factors in more complex situations does not rule out the use of this method. For example, if monitoring is performed during exercise, the equations will include factors such as C₁ (local effects of exercise on the organ), C₂ (systemic effects of exercise), etc. Solution of equations can be achieved by applying more detectors to a variety of sites.

Table 4 below exemplifies hypothetical values for the coefficients used in the model of formulas (3)-(5) above. Optionally or alternatively, other values, scales or coefficients may be used.

TABLE 4

| Organ | A | B |
|---|---|---|
| (muscle) | −1 | −1 |
| (skin) | −1 | +1 |
| (brain) | −1 | +0.01 (~0, negligible) |

Table 4 exemplifies the different effects of different types of organs, namely, while the ‘A’ coefficients (CO₂ factor) for the three listed organs are of the same direction and magnitude (−1), the ‘B’ coefficients (Autonomous system) is the same for muscle and opposite for skin, and negligible for the brain.

A plausible interpretation is that a negative coefficient signifies the fact that resistance is inversely proportional to dilatation, where factors which produce dilatation (high CO₂, sympathetic activity on muscle) increase vessels diameter, thereby increasing flow and decreasing resistance, and vice versa, factors which produce constriction of blood vessels (low CO₂, sympathetic activity on other organs) decrease vessels diameter thereby reducing flow and increasing resistance.

Resistance of blood vessels is related to other haemodynamic parameters that can be measured and evaluated by equipment and methods of the art. For example, PI (Pulsatility Index), RI (Resistivity Index), S/D (Systolic over Diastolic Ratio), or V (blood flow velocities) such as maximal, minimal, mean, and combinations thereof, or other values such as AreaD described above.

Generally, the resistance can be schematically expressed as:

Resistance=g(PI,RI,V,AreaD . . . ) (6)

Where ‘g’ is a function of the haemodynamic parameter or parameters.

For example:

RES(organ)=k(organ)×RI (7)

Where the notation is of the model of formulas (3)-(5) above.

Accordingly, by simultaneously measuring (acquiring) on several sites (tissues) hemodynamic parameters (same parameters or different parameter or combinations thereof) the relative resistance can be calculated such as by formula (7) where the coefficient is obtained by calibration or correlation with two or more organs or tissues.

Having independent values for resistance in organs (e.g. muscle, skin, brain), substituting the independent value into the formulas (3)-(5) above form equations that can be solved and the respective contributions of CO₂ and Autonomic activity factors can be calculated, thereby deriving an evaluation of CO₂ levels.

Substituting in the formulas (3)-(5) above the independently obtained RES values and the coefficients from Table 4, one obtains:

RES(muscle)=(−1)×CO₂+(−1)×Aut+C(muscle)×Oth+D(muscle) (8)

RES(skin)=(−1)×CO₂+(+1)×Aut+C(skin)×Oth+D(skin) (9)

RES(brain)=(−1)×CO₂+0×Aut+C(brain)×Oth+D(brain) (10)

Table 5 below presents a hypothetical analysis of how different conditions, such as listed in Table 3 above, affect the mathematical model of formulas (3)-(5) and respective substituted equations (8)-(9), assuming that the effects of other factors (in addition to CO₂ and Autonomous system) substantially cancel each other as discussed above so that coefficients ‘C’ and ‘D’ do not participate in equations (8)-(9).

TABLE 5

| | | RES | | |
|---|---|---|---|---|
| CO₂ | AUT | Muscle | Skin | Brain |
| −10 max | Max +10 | (10) + (−10) = 0 | (10) + (10) = 20 | (10) + (0) = (10) |
| constriction | Avg 0 | (10) + (0) = (10) | (10) + (0) = (10) | (10) + (0) = 10 |
| low CO₂ | Min (−10) | (10) + (−1* − 10) = 20 | (10) + (−10) = 0 | (10) + (0) = 10 |
| (~20 mmHg) | | | | |
| 0 mid | Max +10 | (0) + (−10) = (−10) | 0 + 10 = 10 | 0 + 0 = 0 |
| diameter | Avg 0 | 0 + 0 = 0 | 0 + 0 = 0 | 0 + 0 = 0 |
| average CO₂ | Min | 0 + 10 = 10 | 0 + (−10) = (−10) | 0 + 0 = 0 |
| (~40 mmHg) | (−10) | | | |
| +10 max | Max +10 | (−10) + (−10) = (−20) | (−10) + 10 = 0 | (−10) + 0 = (−10) |
| dilation | Avg 0 | (−10) + 0 = (−10) | (−10) + 0 = (−10) | (−10) + 0 = (−10) |
| high CO₂ | Min (−10) | (−10) + 10 = 0 | (−10) + (−10) = (−20) | (−10) + 0 = (−10) |
| (~60 mmHg) | | | | |

As based on values in Table 3, Table 5 provides arbitrary sample values for the range of resistance values in different organs. In muscle and skin, the resistance varies between (−20) for lowest resistance (complete dilation) and (+20) for highest resistance (maximal constriction). In the brain, the resistance varies between (−10) for lowest resistance (complete dilation) and (+10) for highest resistance (maximal constriction).

Based on the arbitrary exemplary conditions and results listed in Table 5 above, CO₂ levels can be deduced from RES values using equations (8)-(10), as exemplified in Table 6 below that show muscle and skin resistance parameters and the corresponding CO₂ levels and autonomic activity levels.

In Table 6 only muscle and skin values are exemplified, though it should be noted that using brain values and/or other values may facilitate greater precision than using muscle and skin only.

TABLE 6

| Skin | Muscle | CO₂ level | CO₂ level | AUT activity |
|---|---|---|---|---|
| −20 | 0 | High | 10 | −10 |
| −10 | −10 | High | 10 | 0 |
| −10 | 10 | Normal | 0 | −10 |
| 0 | −20 | High | 10 | 10 |
| 0 | 0 | Normal | 0 | 0 |
| 0 | 20 | Low | −10 | −10 |
| 10 | −10 | Normal | 0 | 10 |
| 10 | 10 | Low | −10 | 0 |
| 20 | 0 | Low | −10 | 10 |

As can be realized from Table 6, distinctive combinations of skin and muscle resistance parameters correlate with distinctive CO₂ and Autonomic activity levels, allowing the calculation of CO₂ levels.

Based on Table 6, FIG. 10 schematically illustrates a diagram describing how CO₂ levels correlate with skin resistance and muscle resistance, according to exemplary embodiments of the invention, where the vertical axis scale 1014 represents the muscle resistance and horizontal axis scales 1012 represents the skin resistance, and where both scales are in a range between (−20) and (+20) in the arbitrary exemplary values discussed above. Line 1002 depicts high level of CO₂ (60 mmHg), line 1004 depicts medium (normal) level of CO₂ (40 mmHg) and line 1006 depicts low level of CO₂ (20 mmHg).

As can be realized from FIG. 10, when skin vascular resistance is in the middle range (0), muscle vascular resistance is inversely proportional to CO₂ which can be directly calculated therefrom. A lowest skin vascular resistance (complete dilatation, (−20)) results from high CO₂ levels with unbalanced autonomic activity, that is, maximal parasympathetic and no sympathetic activity. A maximal skin vascular resistance (maximal constriction, (+20)) results from low CO₂ with unbalanced autonomic activity, that is, maximal sympathetic and no parasympathetic activity.

When the skin vasculature is partly constricted (relative to the middle range of (+10)), a partly constricted muscle vasculature (+10) results from low CO₂ with unbalanced autonomic activity, that is, maximal sympathetic and no parasympathetic activity. A partly dilated muscle vasculature (−10) results from normal CO₂ with balanced autonomic activity. A partly constricted muscle vasculature (+10) results from normal CO₂, and a partly dilated muscle vasculature (−10) results from high CO₂. Other CO₂ levels and/or resistance levels, based on other data may be used.

Using three organs such as muscle, skin and brain as employed in formulas (3)-(5) are used as examples, and a sub-set or larger set of organs or other organs may be used, possibly using a plurality of organs for high fidelity of CO₂ evaluation (e.g. with respect to other methods such a blood sampling) or possibly trading simplicity or convenience (e.g. in emergency) with the fidelity of CO₂ evaluations,

### Special Cases

In some cases the effect of the CO₂ factor is much larger than that of the autonomous system, as well as larger than the other factors, namely:

A(organ)>>B(organ) (11)

A(organ)>>C(organ) (12)

Consequently, formulas (3)-(5) may be represented by one formula of an organ, e.g. skin:

RES(skin)=A(skin)×CO₂+D (14)

Substituting an independent resistance measure equation, such as (7) provides an evaluation of CO₂ level as:

A(skin)=k(skin)*RI (15)

Where ‘RI’ is a resistivity index (or another haemodynamic measure) and the proportionality factor ‘k’ can be calibrated or otherwise determined.

Therefore, in certain cases the multi-signal method can be reduced and simplified to a single signal method.

### Detectors

Standard or specialized sensors may be used for acquiring haemodynamic or related signals from a patient. Following are some viable examples.

1 MHz or 2 MHz PW TCD probes for detecting flow in brain vessels, through skull.

2 MHz or 4 MHz PW probes for detecting flow in Internal Carotid Artery.

4 MHz or 8 MHz PW/CW probes for detecting flow in peripheral arteries, including arteries supplying skeletal muscle.

Photoplethysmography (PPG) probes using IR or NIR (Near Infra-Red) or visible light for detecting flow in skin vasculature (560 nM—green, or 660 nM—Red) and/or muscle vasculature (880 nM—IR).

NIR devices that measure changes (for oxygen saturation) in both skin and brain.

Bioimpedance electrodes for detecting fluid changes that usually reflect blood flow changes in the short term in a variety of organs that may be adapted for skin, muscle and brain.

Laser Doppler probes usually used for evaluation of skin blood flow, also when placed directly on a tissue such as muscle or brain.

Pulse Oximetry sensors (a specific type of PPG) or oxygen saturation (SPO₂) sensors that can provide complementary information for calculation accuracy in extreme values of the CO₂/O₂ range. The raw plethysmographic waveforms generated by these devices, before calculation of SpO₂, can also be used for the general estimation of CO₂ by using the methods as described above.

Pulse oximetry sensors, and/or bioimpedance sensors, specifically adapted for non-invasively measuring blood flow signals of brain tissue.

Tonometric sensors, used for deriving blood pressure changes when placed non-invasively on the skin over representative arteries (or possibly by invasive methods).

ECG, though not a haemodynamic signal per se, can still give information on heart rate which can be used as part of the equations for autonomic activity level.

Other adequate new or customized detectors or other equipment suitable for detecting and acquiring haemodynamic signals or related signals can be used, optionally with some modifications or adjustments, preferably as non-invasive sensors.

### System (Apparatus)

In some embodiments of the invention the detector or detectors are connected to or integrated with electronic and/or electrical and/or mechanical components and/or other components (e.g. chemicals such that change color due to heat), providing a system for evaluation and/or monitoring of CO₂ levels of a patient by implementing one or more of the methods such as described above or variation and/or part thereof.

In some embodiments of the invention, the system performs additional activities such as derivation and calculations of other parameters of the patient (e.g. heart rate, respiration rate), archiving, trending, correlations with past measurements of the patient or other patients, or linkage with other systems.

In some embodiments of the invention the system comprises or is linked with one or more processors. In some embodiments, the system comprises or is integrated with or linked with a medium comprising or storing a program or programs, optionally with auxiliary data, that implements one or more algorithms and/or procedures and optionally with a medium for storing data. The tasks performed by the system with the processor and program comprise acquiring and processing the acquired signals, performing the computations to obtain a value of the CO₂ level of the patient, and optionally other tasks such as calibration or control and supervision of components of the system (e.g. of a sensor), or interaction with the user (operator) or obtaining some other parameters of the patient.

Typically, in some embodiments, the system operates continuously and monitors CO₂ level in real-time (at least relative to the approximate respiration rate of the patient).

In some embodiments of the invention, the system comprises built-in (or remote) display and/or a printer to provide readout of CO₂ level or other parameters and optionally of waveform of the acquired or conditioned signals (e.g. for system checking). Optionally or additionally, the system comprises other apparatus to provide the evaluation of CO₂ level or other values, such as a voice-generation apparatus as a readout medium. Optionally or additionally, the system comprises user interface comprising elements such as buttons or sliders and/or indicators (e.g. LEDs) and/or graphical interface. The user interface is used for tasks such as calibration, control (e.g. on/off), or setting operation modes. Optionally, the system comprises buzzer or other alarm equipment (e.g. vibrations) to notify about physiological conditions and/or system malfunction or bad contact or connection of the sensor to the patient.

In some embodiments of the invention, the system comprises components (e.g. readout with limits or zones indications or alarm buzzer) such as to provide feedback to the patient, optionally assisting the patient to regulate the respiration and/or CO₂ level.

In some embodiments of the invention, the system comprises components (to provide linkage or feedback to another device, such as an artificial ventilator, optionally assisting the second device to regulate the respiration and/or CO₂ level. In some embodiments, the linkage is by a communication link (e.g. cable or wireless) or the linkage can be a visual and/or audible indication that alerts personnel to activate the second device.

In some embodiments of the invention, the system is a portable system, optionally sufficiently small and light for wearing on the body of the patient (e.g. an ambulatory patient), such as on a belt or a wrist and is, optionally, battery operated.

It should be noted that attaching electrodes or other external sensors to or proximate to the skin, as may be used in conjunction with the system described above, can provide an effective method of monitoring patients in, for example, emergency or ambulatory situations.

It is generally assumed herein that an appropriate power supply is used for the system operation.

FIG. 12 schematically illustrates a diagram of a system 1200 for CO₂ evaluation illustrating with arrows the main control linkages between the components thereof, according to exemplary embodiments of the invention.

System 1200 comprises or is connected to a sensor 1202 which is attached to the patient (1304) being monitored. Optionally, system 1200 comprises or is connected to additional sensors exemplified as 1202a and 1202b and marked with dashed outline (collectively sensor 1202) wherein the additional sensors are attached to other tissues or organs of the patient. Typically and preferably, sensors 1202 are attached on the skin of the patient or approximate to the skin (non-invasive detection), while in some embodiments one or more of sensors 1202 are used subcutaneously or in a vein or artery.

The system operation is carried out by a processor (or processors) 1206 according to a program or programs and data stored in memory 1210 under the control of a user interface 1208. Memory 1210 typically comprises read-only memory and/or read/write memory. The output of sensor 1202 is collected (acquired) via input ports of the processor (or other ports) into a buffer 1204 for storing the raw data that is further processed. Optionally, buffer 1204 is comprised in memory 1210 or in a module of processor 1206. System 1200 optionally comprises a buzzer 1214 representing also any other alarm equipment or mechanism.

### Operation Overview

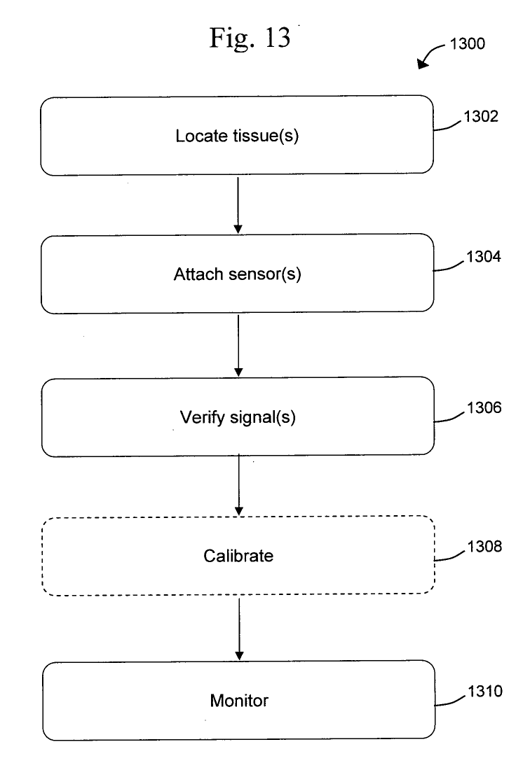

FIG. 13 illustrates a flowchart 1300 outlining actions for user operation involved in evaluating CO₂ level of a patient, according to exemplary embodiments of the invention. In the following discussion reference to system 1200 of FIG. 12 is implied as a non-limiting example.

Suitable tissue or tissues of the patient for using sensor or sensors 1202 are located (1302) and optionally prepared, for example, a patch or region of skin to be used is located and cleaned.

Sensor (or sensors) 1202 are attached to the patient, optionally mechanically secured to ensure sufficient and stable contact, for example, by an elastic band or strap with a fastener such as buckle or hooks-and-loops pair.

Using user interface 1208 (or as a default operation upon connecting sensor 1202), system 1200 begins to acquire signals which are verified for acceptability (1306). For example, the signals are visually verified by showing on display 1212 the signal with lower and/or lower acceptable limits and if the signal is outside the limits, or the signal is noisy or irregular, the sensor and/or contact thereof to the patient should be checked. Optionally or additionally, in some embodiments, the signals stored in buffer 1204 are compared by processor 1206 to a template or templates of an appropriate signal stored in memory 1210 (e.g. typical template and/or upper and lower limits templates) and/or the quality of the signal is assessed for regularity and noise, and processor 1206 alarms the operator by display 1212 and/or buzzer 1214 in case of non-acceptable signals.

Having acquisition of appropriate signals, system 1200 is calibrated (1308) if necessary (e.g. system 1200 may be already calibrated, or possesses automatic calibration capability). Calibration may be carried out by acquiring CO₂ level from another source, for example, capnograph or using kit for blood sample CO₂ evaluation or intra-arterial CO₂ analyzer. Optionally or alternatively, the calibration may be carried out by processor 1206 optionally with data in memory 1210 using matching or convergence procedures to reach plausible CO₂ values.

When the signals are acceptable and the system 1200 is calibrated, system 1200 is set, typically by user interface 1208, to start monitoring (1310). Optionally, by user interface 1208 an operation mode is set, such as continuous evaluation, periodic evaluation, what to display, whether other parameters are obtained and displayed, etc.

Optionally, using user interface 1208 operational limits are set so that system 1200 activates buzzer 1214 and/or displays notification on display 1212 if the limits are breached.

In some embodiments, system 1200 supervises the acquired signals for acceptability (see also above) and in case of insufficient signal quality system 1200 activates buzzer 1214 and/or displays notification on display 1212

### Advantages

Possible and/or probable advantages of monitoring CO₂ level, particularly non-invasively and more particularly with portable light-weight apparatus, is a fast and simple operation which can be important in emergency cases or for long-term monitoring of CO₂ akin to Holter recorder.

Another possible advantage is evaluating CO₂ levels directly correlated with arterial CO₂ and that in a non-invasive manner. Current measurements using a capnograph measure End-Tidal-CO₂ values which reflect CO₂ values within the lungs so that when there is a pause in breathing (apnea), for example, the capnograph cannot measure and provide CO₂ values. On the other hand, by using the methods and equipment such as described above CO₂ and evaluation based on the heart and vascular activity can be continuously provided.

### General

The following non-limiting characterizations of terms are applicable in the specification and claim unless otherwise specified or indicated in or evidently implied by the context, and wherein a term denotes also variations, derivatives, inflections and conjugates thereof.

The terms ‘processor’ or ‘computer’, beyond the ordinary context of the art, denote any deterministic apparatus capable to carry out a provided or an incorporated program and/or access and/or control data storage apparatus and/or other apparatus such as input and output ports.

The terms ‘software’, ‘program’, ‘software procedure’ (‘procedure’) or ‘software code’ (‘code’) may be used interchangeably, and denote one or more instructions or directives or circuitry for performing a sequence of operations that generally represent an algorithm and/or other process or method. The program is stored in or on a medium (e.g. RAM, ROM, disk, etc.) accessible and executable by an apparatus such as a processor or other circuitry.

The processor and program may constitute the same apparatus, at least partially, such as an array of electronic gates (e.g. FPGA, ASIC) designed to perform a programmed sequence of operations, optionally comprising or linked with a processor or other circuitry.

The terms ‘about’, ‘close’, ‘approximate’, ‘practically’ and ‘comparable’ denote a respective relation or measure or amount or quantity or degree yielding an effect that has no adverse consequence or effect relative to the referenced term or embodiment or operation or the scope of the invention.

The terms ‘substantial’, ‘considerable’, ‘significant’, ‘appreciable’ (or synonyms thereof) denotes a measure or extent or amount or degree which encompass most or whole of a referenced entity, or is sufficiently large or close or effective or important relative to a referenced entity or with respect the referenced subject matter.

The terms ‘negligible’, ‘slight’ and ‘insignificant’ (or synonyms thereof) denote, a sufficiently small respective relation or measure or amount or quantity or degree to have practical consequences relative to the referenced term and on the scope of the invention.

The terms ‘similar’, ‘resemble’, ‘like’ and the suffix ‘-like’ denote shapes and/or structures and/or operations that look or proceed as, or approximately as the referenced object.

The terms ‘constant’, ‘uniform’, ‘continuous’, ‘simultaneous’ and other seemingly definite terms denote also close or approximate respective terms.

The terms ‘vertical’, ‘perpendicular’, ‘parallel’, ‘opposite’, ‘straight’ and other angular and geometrical relationships denote also approximate yet functional and/or practical, respective relationships.

The terms ‘preferred’, ‘preferably’, ‘typical’ or ‘typically’ do not limit the scope of the invention or embodiments thereof.

The terms ‘comprises’, ‘comprising’, ‘includes’, ‘including’, ‘having’ and their inflections and conjugates denote ‘including but not limited to’.

The term ‘may’ denotes an option which is either or not included and/or used and/or implemented, yet the option comprises a part of the invention.

Unless the context indicates otherwise, referring to an object in the singular form (e.g. ‘a thing” or “the thing”) does not preclude the plural form (e.g. “the things”).

The present invention has been described using descriptions of embodiments thereof that are provided by way of example and are not intended to limit the scope of the invention or to preclude other embodiments. The described embodiments comprise various features, not all of which are necessarily required in all embodiments of the invention. Some embodiments of the invention utilize only some of the features or possible combinations of the features. Alternatively and additionally, portions of the invention described or depicted as a single unit may reside in two or more separate entities that act in concert or otherwise to perform the described or depicted function. Alternatively and additionally, portions of the invention described or depicted as two or more separate physical entities may be integrated into a single entity to perform the described/depicted function. Variations related to one or more embodiments may be combined in all possible combinations with other embodiments.

When a range of values is recited, it is merely for convenience or brevity and includes all the possible sub-ranges as well as individual numerical values within that range. Any numeric value, unless otherwise specified, includes also practical close values enabling an embodiment or a method, and integral values do not exclude fractional values. A sub-range values and practical close values should be considered as specifically disclosed values.

In the specifications and claims, unless particularly specified otherwise, when operations or actions or steps are recited in some order, the order may be varied in any practical manner.

Terms in the claims that follow should be interpreted, without limiting, as characterized or described in the specification.

1. A method for evaluating CO₂ level in the blood of a patient, comprising:
(a) detecting from the patient's body at least one haemodynamic signal from at least one tissue or part thereof;
(b) processing the at least one haemodynamic signal to derive a value related to the CO₂ level in the blood of the patient; and
(c) determining an evaluation of CO₂ level of the patient based on a relation of the derived value to CO₂ level in the blood of the patient.

2. The method according to claim 1, wherein detecting is performed non-invasively.

3. The method according to claim 1, wherein the at least one haemodynamic signal from at least one tissue or part thereof constitutes one signal from one tissue or part thereof.

4. The method according to claim 1, wherein the at least one haemodynamic signal from at least one tissue or part thereof constitutes a plurality of signals from a plurality of similar tissues or parts thereof.

5. The method according to claim 4, wherein the plurality of signals are detected simultaneously.

6. The method according to claim 4, wherein the similar tissues are disjoint skin regions.

7. The method according to claim 1, wherein the at least one haemodynamic signal from at least one tissue or part thereof constitutes a plurality of signals from one tissue or part thereof.

8. The method according to claim 7, wherein the plurality of signals are detected simultaneously.

9. The method according to claim 7, wherein the one tissue or part thereof is a skin region.

10. The method according to claim 1, wherein the at least one haemodynamic signal from at least one tissue or part thereof constitutes a plurality of signals from a plurality of different tissues or parts thereof.

11. The method according to claim 10, wherein the plurality of signals are detected simultaneously.

12. The method according to claim 10, wherein the plurality of different tissues comprises at least one tissue selected from skin, muscle or brain.

13. The method according to claim 10, wherein the plurality of different tissues comprises at least two tissues selected from skin, muscle or brain.

14. The method according to claim 1, wherein processing comprises identifying a region of the at least one signal, or a derivative thereof, by which a value functionally related to CO₂ level of the patient is derived.

15. The method according to claim 14, wherein identifying a region comprises analyzing a temporal derivative, or a combination thereof, of the at least one signal or a derivative thereof.

16. The method according to claim 14, wherein a value functionally related to CO₂ level of the patient is derived by integrating the temporal derivative, or a combination thereof, about the region.

17. The method according to claim 14, wherein the value functionally related to CO₂ level of the patient is linearly related to CO₂ level of the patient.

18. The method according to claim 1, wherein processing comprises:
(a) defining a model of a haemodynamic parameter based on a plurality of signals from a plurality of different tissues or parts thereof; and
(b) substituting in the model at least one separately acquired haemodynamic parameter thereby deriving a value related to the CO₂ level of the patient.

19. The method according to claim 1, wherein the value related to the CO₂ level of the patient constitutes the evaluation of CO₂ level of the patient.

20. An apparatus for evaluating CO₂ level in the blood of a patient, comprising:
(a) at least one detector on the patient's body for detecting at least one haemodynamic signal from an at least one tissue or part thereof; and
(b) a processor and a program for deriving an evaluation of the CO₂ level of the patient based on the at least one haemodynamic signal.

21. The apparatus according to claim 20, further comprising an apparatus for providing at least the evaluation of the CO₂ level in the blood of the patient.

22. The apparatus according to claim 20, wherein the evaluation of the CO₂ level is provided continuously in real-time.

23. The apparatus according to claim 20, wherein the at least one detector is non-invasive.

24. The apparatus according to claim 20, wherein the apparatus is sufficiently small and lightweight for wearing by the patient.

25. The apparatus according to claim 20, wherein the apparatus is sufficiently mobile to be worn by an ambulatory patient.

26. (canceled)
